(19) United States
(12) Patent Application Publication
(10) Pub. No.: US 2012/0046369 A1
(43) Pub. Date: Feb. 23, 2012

(54) Methods for enhancing the stability of foods, beverages, and cosmetics using natural products derived from non-allergenic proteinaceous sources

(75) Inventors: Roger Nahas, Portage, MI (US); Peter Collins Vanalstyne, Paw Paw, MI (US); Anita E. Uhlir, Paw Paw, MI (US); Donald Berdahl, Lawton, MI (US); James Barren, Kalamazoo, MI (US)

(73) Assignee: KALAMAZOO HOLDINGS, INC.

(21) Appl. No.: 13/136,718

(22) Filed: Aug. 9, 2011

Related U.S. Application Data
(60) Provisional application No. 61/401,255, filed on Aug. 10, 2010.

(51) Int. Cl. A61K 8/64; C07K 2/00; C09K 15/34; A23L 3/3454; A23L 2/44

(52) U.S. Cl. 514/773; 426/542; 252/398; 530/300

(57) ABSTRACT

The present invention relates to compositions with effective metal chelating activity and utility as natural antioxidants in food, beverages, nutritional supplements and cosmetics. The compositions of the present invention may be prepared by enzymatically hydrolyzing hypoallergenic protein isolates derived from vegetables, for example yellow pea (Pisum sativum), and/or grains using specific enzymes.

### FIELD OF THE INVENTION

The instant invention relates to compositions and methods for enhancing the stability of foods, beverages, nutritional supplements and/or cosmetics by incorporating into them effective amounts of natural metal chelating antioxidant compositions derived from vegetables and/or grains. Moreover, the instant method for enhancing the stability of foods may, optionally, further comprise incorporating one or more chelating or non-chelating antioxidant components derived from edible herbs, spices, fruits, vegetables and/or grains, and which may further be combined with one or more synthetic food grade antioxidants. Enhanced stability includes flavor stability, color stability, textural stability and/or component stability (such as lipid, vitamin, carotenoid, protein or other constituent).

Moreover, the present invention relates to processes for preparing metal chelating or sequestering antioxidant compositions with specific activities and solubility characteristics tailored to distribute the metal chelating and other antioxidant components within the foods, beverages, nutritional supplements or cosmetics where the metal chelators/antioxidants operate most effectively. The present invention further relates to foods, beverages, nutritional supplements and cosmetics treated with the inventive compositions.

### BACKGROUND OF THE INVENTION

### Description of the State of the Art

### Oxidation in Foods and Antioxidants

Substances that serve to protect foods from the deleterious effects of oxidation are commonly added to foods and are called antioxidants or stabilizers. These substances can be naturally or synthetically derived, although consumers generally prefer those materials derived from natural sources. The performance of a given antioxidant is dependent upon many things, including its chemical nature (stability, reactivity, functionality and the like) and its physical properties (volatility, solubility, polarity and the like). Antioxidant substances can have different modes of action, interfering with oxidation processes in a number of ways. Substances function as antioxidants if they:

Disrupt the oxidation mechanism by reacting with free radical intermediates (radical scavengers).

React preferentially with oxygen, removing it from the environment of the substrate being stabilized (oxygen scavengers).

Absorb, and render less harmful, energy from incident radiation or energy from excited chemical species (quenchers).

Reduce and thereby regenerate oxidized antioxidants (antioxidant regenerators).

Reduce peroxidic intermediates to non-radical products (secondary antioxidants).

Sequester and lessen the activity of metal initiators of oxidation (metal chelators).

Some of the most commonly used and most effective metal chelating additives in foods, beverages, cosmetics and nutritional supplements are derivatives of the synthetic compound ethylenediamine tetraacetic acid (EDTA). The structure of EDTA makes it a very powerful metal chelator. EDTA is particularly useful in stabilizing oil and water containing emulsion systems, such as mayonnaise, salad dressings, emulsified beverages, and the like.

Since EDTA has many industrial applications, it has become widespread in the environment and is the most abundant man-made compound in many European surface waters. Although the isolated molecule does not present a risk of bioaccumulation, the ligand-metal complexes may significantly increase the bioavailability of extremely dangerous heavy metals (Oviedo and Rodriguez, 2003). Because of these concerns, and the consumer preference for natural as opposed to synthetic additives, there is a need to find a natural, preferably GRAS (Generally Recognized As Safe) replacement for this important and highly functional food additive.

### Methods for Assessing the Activity of Antioxidants and Model Systems

The present invention relates to a method for stabilizing foods, beverages, nutritional supplements and cosmetics, using as one component, hydrolyzed vegetable protein with metal chelating properties. One method for measuring the metal chelating strength of a substance is the so-called ferrozine assay. Ferrozine (3-(2-pyridyl)-5,6-diphenyl-1,2,4-triazine-4′-4″-disulfonic acid, sodium salt) is commonly used to assess the potential of materials to chelate Fe(II). Ferrozine forms a colored complex with Fe (II) with a maximum absorbance at 562 nm (Carter, 1971). The potency of the extracts or pure compounds to bind ferrous ions is assessed by their competition with ferrozine resulting in a decrease in the formation of the colored complex. The degree of color fading is assessed by measuring the absorbance at 562 nm and correlated to the strength by which the chelator binds to the metal.

Model systems that are simpler representations of foods, beverages, nutritional supplements and cosmetics can also be used to test the performance of antioxidant compositions. Food systems that contain polyunsaturated fats are subject to lipid oxidation leading to deterioration of food quality and formation of off-flavors. Oxidation can be monitored by measuring the primary oxidation products (hydroperoxides) as well as secondary oxidation products (aldehydes and ketones). The hydroperoxides monitoring test is a spectroscopic method that allows the assessment of the oxidative stability of a bulk oil system or oil and water emulsion system and the efficacy of antioxidant treatments by measuring the hydroperoxides in a system in cumene hydroperoxide equivalents, via the conversion of iron (II) to iron (III) (Bou et al., 2008) or by simply monitoring the emulsion absorbance at 234 nm which is the absorbance of the conjugated dienes hydroperoxides. Most of the emulsion models established to mimic food systems and used as a matrix to test the performance of various antioxidants consist of oil-in-water emulsions (O/W). Foods containing O/W emulsions include mayonnaise, milk, cream, etc. Water-in-oil emulsions (W/O), wherein the oil (the continuous phase) surrounds droplets of water (the discontinuous phase), include butter and margarine, for example.

### Hydrolyzed Proteins as Food Additives

Hydrolyzed proteins (from vegetable and animal sources) are commonly used in foods to enhanced functionality and properties such as improved foaming, better “mouth feel”, flavoring, emulsification capability and nutritional fortification. Hypo-allergenic proteins constitute highly desirable sources of protein hydrolysates due mainly to the absence of an allergen declaration on the label of food products containing these protein hydrolysates. None of the marketed hydrolyzed vegetable proteins are recognized as a food preservative/antioxidant functioning at a very low dose, much lower than the dose required for the above mentioned functional properties.

### The Advantage of the Hypo-Allergenic Pea Protein Hydrolysate as a Food Additive

Pea protein is one of the highly desirable sources of protein hydrolysates because the hydrolysates are hypoallergenic. In addition, several studies showed that hydrolyzed pea protein carries several allergenic and immuno-related benefits over the unhydrolyzed protein (Szymkiewicz and Jedrychowski, 2008).

U.S. Pat. No. 5,520,935 claims a method for producing a pea protein hydrolysate for use as a dietetic supplement. The method of the invention is described to provide palatable pea protein products for dieticians in hospitals and homes for elderly people, as well as for manufacturers of dietetic products, and which protein products are also intended for athletes.

There is a growing interest in hydrolyzed pea protein as a health beneficial functional ingredient. Humiski and Aluko (2007) compared the effect of using different enzymes (ALCALASE® (Protease; Subtilisin), FLAVOURZYME® (aminopeptidase), papain, trypsin, and α-chymotrypsin) on the antioxidant activity of the resulting protein hydrolysates for use as a therapeutic. The antioxidant activity was measured by the DPPH (2,2-diphenyl-1-picrylhydrazyl) assay (radical scavenging activity) and evaluated inhibition of angiotensin converting enzyme (ACE) activity. Bitterness of the protein hydrolysates was also evaluated. The use of the enzymes, papain and α-chymotrypsin, in preparing protein hydrolysates was recommended because the resulting hydrolysates were less bitter and the hydrolysates demonstrated a greater effect on inhibiting ACE.

In 2008, a study compared three different proteins hydrolyzed with various enzymes, for ACE inhibition, Calmodulin-binding peptides, copper chelating and antioxidant peptides. In many of these tests, the crude hydrolysates were purified by ultra-filtration and, optionally, by further fractionation (Aluko, 2008). The fractions were tested to determine whether the smaller-sized peptides are more bioactive than the larger sized peptides. The investigators concluded the following: “One of the limiting factors in the utilization of food proteins as sources of therapeutic peptides is the low potency of the initial hydrolysate fractions when compared to available drugs. Even though processing methods such as ultra-filtration and column chromatography can be used to enrich the protein hydrolysates into very potent fractions, the economic viability of such processes is doubtful. Therefore, efforts must continue in developing more efficient hydrolytic and cheaper separation or purification methods that are compatible with industrial production practices and have commercial viability”. Therefore, it would be surprising if hydrolyzed pea protein would be functional at low doses, especially without purification.

Pownall et al. (2010) hydrolyzed pea protein with an enzyme, thermolysin, which specifically cleaves at hydrophobic amino acids residues in the protein. The hydrolysate was purified by ultra-filtration techniques to obtain highly water soluble peptide fractions with lower molecular weights than the starting crude peptide fractions. Finally, the peptide fractions were purified further by HPLC fractionation to obtain five fractions, which were then spray dried. After the purification and isolation, the fractions were tested for radical scavenging activity, H₂O₂ scavenging, metal chelating and reducing power, and inhibition of linoleic acid oxidation. Pownall et al. concluded that “the enzymatic pea seed hydrolysates could be used as potential ingredients to formulate functional foods and nutraceutical products”.

### Need for Natural Preservatives and Potential Food Applications and Intent of the Invention

Naturally-derived antioxidants are used as stabilizers in many food, beverage, nutritional supplements and cosmetics products. However, there are many products and ingredients that are highly oxidatively unstable and for which the current state of the art antioxidants are insufficient to provide the degree of increased oxidative stability required or desired. It is the aim of the present invention to provide methods and compositions to improve the stability products that are difficult to stabilize with existing naturally-derived products, using an effective, hypo-allergenic, vegetable protein derived product, obtained from a simple process that utilizes minimal purification and clean-up steps.

### Dressings

Much of the commercial salad dressings sold around the world are stabilized with derivatives of the synthetic antioxidant, EDTA. EDTA is a very powerful chelating agent and is very effective in preserving the flavor of mayonnaise in storage. In Germany, EDTA is not allowed in mayonnaise. Absent the ability to use this highly effective stabilizer to obtain sufficient shelf life, German mayonnaise with must be manufactured with oils that are inherently more stable than the oils often used in other countries, namely oils that are relatively more saturated. The use of more saturated fats runs counter to the desire to include more unsaturated fats in the diet. Thus, there is a need to make and sell mayonnaise that incorporates more highly unsaturated and less inherently stable oils. Indeed, mayonnaise preparations containing highly unsaturated fish and algal-derived oils are desired for their health benefits. The stabilizing agents currently allowed in dressings according to German regulations are not sufficiently effective to stabilize mayonnaise made with oils having higher levels of unsaturation. The present invention provides materials and methods to enhance the stabilization of mayonnaise and related dressings and the like, beyond what is now practiced in the art.

### Coffee Creamers, Milk and Powdered Milk

Milk, dairy- and non-dairy coffee creamers, and oil containing emulsion beverages are one of the most commonly used oil in water food and beverage emulsions. They suffer from oxidative effect on flavor and overall quality due to the faster rate of oxidation, generally attributed to the large contact surface of the oil with water. Powdered milk also is affected by oxidative deterioration of sensory quality due to the effect of spray-drying on the heat induced oxidation, and the oxidation of the fat during storage. The present invention provides materials and methods to enhance the stabilization of oil-in-water emulsions such as dairy products, creamers and the like, beyond what is now practiced in the art.

### Cured Meats

Cured meats are subject to oxidation processes that result in the loss of desirable flavors, the formation of off-flavors, the loss of desirable cured meat pigment color, and the formation of undesirable colors, among other effects that cause a decrease in the shelf life of the product. Cured meats are also subject to the growth of bacteria, yeasts and molds that also shorten the shelf life of the product. The purpose of this present invention is to provide materials and methods to enhance the oxidative stability of cured meats, and limit the quality damage on flavor, color and shelf-life.

Fish, Algal, and Vegetable Oils with High Levels of Unsaturation

Highly unsaturated oils are very susceptible to oxidation and they are, therefore, difficult to incorporate into food, beverage, nutritional supplement and cosmetic products. Unsaturated oil emulsions are particularly difficult to stabilize. The purpose of the present invention is to provide materials and methods to enhance the stabilization of fish, algal and vegetable oils containing high levels of unsaturation, and the like, beyond what is presently achievable.

### Frying Oil and Fried Foods

The frying process subjects the frying oil and the article being fried to severe oxidative stress. Current state of the art antioxidants, both natural and synthetic, fail to provide the desired stabilizing effects. The purpose of the present invention is to provide materials and methods to improve the shelf life and quality of frying oils and of fried foods.

### Potted Meats

Meat products, especially, including baby food preparations, which are retorted in metal, glass or plastic containers, often suffer oxidative damage leading to off-color formation, particularly at the surface of the product. The development of off-flavors can also occur during the retort process and in the period during which the product is stored prior to use. It is a further purpose of this invention to provide materials and methods to stabilize potted meat products against oxidation resulting in flavor and color changes.

### Coffee and Coffee Concentrates

Coffee extracts or concentrates are replacing freshly brewed coffee in many retail settings. Freshly brewed coffee and coffee extracts or concentrates are susceptible to oxidative process leading to unwanted flavor changes. It is a further purpose of this invention to provide materials and methods to stabilize coffee and coffee extracts or concentrates against oxidatively induced flavor changes.

### Beer and Malt Beverages

Beer and other malt beverages undergo undesirable flavor changes as a result of oxidative processes during the brewing process and in storage. The purpose of the present invention is to provide materials and methods to increase the flavor stability and shelf life of beer and malt beverages.

### Natural and Artificial Coloring Agents

Many natural and synthetic coloring agents are oxidatively unstable. Color loss in meat, beverages, foods, cosmetics and in the coloring compositions, themselves, accompanies the oxidation of these materials. It is a further purpose of this invention to provide materials and methods to stabilize natural and artificial coloring agents such as anthocyanins, carotenoids, xanthophylls, capsanthin, capsorubin, lutein, zeaxanthin, bixin, norbixin, astaxanthin, beta-cryptoxanthin, lycopene, beta-carotene, alpha carotene, FD&C colors, chlorophylls, myoglobin, oxymyoglobin, nitrosomyoglobin, carboxymyoglobin, carmine, carminic acid, turmeric extract, curcumin, annatto extract, paprika extract, carrot extract, tomato extract, algal extracts, beet extract, hyacinth extracts, gardenia extracts, spinach extracts and the like against oxidation resulting in color and flavor changes in foods, beverages, nutritional supplements, cosmetics or in the natural and artificial coloring agents, themselves.

### Irradiated Meats

Treatment of fresh meat, poultry and seafood with incident radiation as described in U.S. Pat. No. 6,099,897, herein incorporated by reference in its entirety, induces unwanted oxidative changes in the color, flavor and storage stability in the final irradiated product. The purpose of the present invention is to provide materials and methods to stabilize irradiated meat, poultry and fish products against oxidation resulting in flavor and color changes beyond what is now practiced in the art.

### BRIEF SUMMARY OF THE INVENTION

It has now been discovered that compositions with effective metal chelating activity and utility as antioxidants in food, beverages, nutritional supplements and cosmetics can be prepared by enzymatically hydrolyzing hypoallergenic protein isolates derived from vegetables, for example yellow pea (Pisum sativum), and/or grains with specific enzymes.

The present invention relates to the surprising metal chelating characteristics of antioxidant compositions derived from yellow pea (Pisum sativum), which hydrolysate compositions are unexpectedly effective at very low doses (0.001-0.25%). The antioxidative compositions can be obtained, even without any ultra-filtration or peptide fractionation processing of the protein hydrolysate.

The present invention further relates to highly effective antioxidant compositions made up of combinations of metal chelating elements derived from herbs, spices, fruits and/or vegetables, optionally, together with radical scavengers, oxygen scavengers, secondary antioxidants, quenchers and/or antioxidant regenerators derived from natural and/or synthetic sources.

The present invention thus provides methods for stabilizing foods, beverages, cosmetics and/or nutritional supplements by the application of vegetable and/or grain-derived metal chelating compositions, optionally containing additional natural and/or synthetic antioxidants to the said food, beverage, cosmetic and or nutritional supplement, in an amount sufficient to have a measurable stabilizing effect.

The present invention further provides stabilized foods, beverages, cosmetics and/or nutritional supplements comprising a food, beverage, cosmetic and/or nutritional supplement, together with a stabilizing composition consisting of metal chelating elements derived vegetables and/or grains, optionally combined with synthetic; and/or natural antioxidants of the radical scavenger, oxygen scavenger, secondary antioxidant, quencher and/or antioxidant regenerator types.

### OBJECTS OF THE INVENTION

It is a general object of the present invention to provide metal-chelating or metal-sequestering antioxidant compositions, derived from edible vegetables and/or grains, for incorporating into foods, beverages, nutritional supplements and cosmetics to enhance the stability of the food, beverage or cosmetic. It is also an object of this invention to provide methods for preparing hypoallergenic antioxidant, stability-enhancing compositions.

This invention provides a method for stabilizing the fresh flavor and preventing the formation of off-flavors in dairy products and non-dairy corresponding products (where the animal fat is substituted with vegetable fat), salad dressings and other oil-in-water emulsion-based food systems by treating these materials at some stage in their production with an effective amount of a metal chelating antioxidant composition derived from hypoallergenic isolated protein obtained from vegetable and/or grain matter, for example, hydrolyzed yellow pea protein, optionally containing one or more non-chelating antioxidant components also derived from edible herbs, spices, fruits, vegetables and/or grains, and/or further optionally combined with one or more synthetic food grade antioxidants; in a manner which does not impact the taste or color of the foods.

This invention provides a method for stabilizing the fresh flavor and color and preventing the formation of off-flavors and off-colors in cured meats, including ham, bacon, salt pork, sausage, kippered herring, beef jerky, salami, summer sausage, cold cuts, bologna, pastrami, pepperoni, corned beef, roast beef, hot dogs, dried beef, bratwurst, polish sausage, barbecued pork, pork loin, beef brisket, salmon, liverwurst, pork char sui, prosciutto, culatello, lomo, coppa, bresaola, lardo, guanciale, mocetta, qadid, and the like, by incorporating into these materials at some stage in their production, an effective amount of a metal chelating antioxidant composition derived from hypoallergenic isolated protein obtained from vegetable and/or grain matter, for example, hydrolyzed yellow pea protein and optionally, containing one or more non-chelating antioxidant components also derived from edible herbs, spices, fruits, vegetables and/or grains, and/or, optionally, combined with one or more synthetic food grade antioxidants.

This invention provides a method for stabilizing the fresh flavor and preventing the formation of off-flavors in frying oils, and in the foods fried in the oil, by treating the frying oil prior to or during the frying operation with an effective amount of a metal chelating antioxidant composition derived from hypoallergenic isolated protein obtained from vegetable and/or grain matter, for example, hydrolyzed yellow pea protein, and optionally containing one or more non-chelating antioxidant components derived from edible herbs, spices, fruits, vegetables and/or grains, and/or, optionally, combined with one or more synthetic food grade antioxidants.

This invention provides a method for slowing the rate of oxidation, stabilizing the fresh flavor and preventing the formation of off-flavors in fats and oils containing polyunsaturated lipids by treating these materials with an effective amount of a metal chelating antioxidant composition derived from hypoallergenic isolated protein obtained from vegetable and/or grain matter, for example, hydrolyzed yellow pea protein, and optionally containing one or more non-chelating antioxidant components derived from edible herbs, spices, fruits, vegetables and/or grains, and/or, optionally, combined with one or more synthetic food grade antioxidants.

This invention provides a method for slowing the rate of oxidation, stabilizing the fresh flavor and preventing the formation of off-flavors in extruded human and animal foods by incorporating into them at some stage in their production or use, an effective amount of a metal chelating antioxidant composition derived from hypoallergenic isolated protein obtained from vegetable and/or grain matter, for example, hydrolyzed yellow pea protein. The antioxidant composition may, optionally, contain one or more non-chelating antioxidant components also derived from edible herbs, spices, fruits, vegetables and/or grains, and/or, optionally, combined with one or more synthetic food grade antioxidants.

Many of the antioxidant, metal chelating compositions of the invention also surprisingly show anti-microbial activity in the foods into which they are incorporated, by slowing or preventing the growth of microorganisms.

Other objects, features and advantages of the present invention will become apparent as one reads carefully through the descriptive examples that are not in any way limiting.

### BRIEF DESCRIPTION OF THE FIGURES

FIG. 1. Representative results of inhibition of oxidation in oil in water emulsions.

FIG. 2. Representative results of inhibition of oxidation in margarines.

FIG. 3. Representative results of inhibition of oxidation in powdered milk.

FIG. 4. Representative results of inhibition of oxidation in cereals and extruded foods.

### DETAILED DESCRIPTION OF THE INVENTION

We have found that antioxidative, natural metal chelating compositions useful for stabilizing foods, cosmetics, beverages and nutritional supplements can be prepared from protein isolates by enzymatic hydrolysis. The vegetables and grains that serve as sources of these protein hydrolysates are preferably high in hypoallergenic proteins. Such hypoallergenic protein sources include yellow pea, potatoes, barley, canola, rapeseed, alfalfa and fabaceous bean. Moreover, the aforementioned hypoallergenic protein sources have the advantage of being consumer friendly and are economically efficient due to their abundance and high yield.

Hypoallergenic hydrolyzed proteins may be obtained from spice, herb, fruit and/or vegetable matter that contain low levels of protein such as allspice, anise, star anise, caper, caraway, cardamom, Capsicum pepper, cinnamon, clove, coriander, cumin, curry, dill, fennel, ginger, mace, nutmeg, marjoram, mustard, paprika, black pepper, white pepper, saffron, sage tarragon, thyme, turmeric, rosemary, galangal, balm, basil, grains of paradise, bay, basil, celery, licorice, mint, mistletoe, parsley, peppermint, valerian, vanilla, carrot, tomato and the like.

Antioxidant protein hydrolysates may be also obtained from hypoallergenic sources such as corn and rice.

Less preferably, antioxidative protein hydrolysates may be obtained from allergenic sources such as soybean, wheat, tree nuts and peanuts. Also less preferably, antioxidative protein hydrolysates may be obtained from animal protein sources such as milk (whey and casein), fish, shellfish and eggs. The antioxidant substances extracted from these spices, herbs, vegetables and/or fruits can be combined to form more complex antioxidant compositions. The antioxidant compositions can be obtained in a variety of ways.

Methods of obtaining the metal chelating compositions of the invention include the steps of isolating the protein from its vegetable and/or grain source, and enzymatic hydrolysis, thereby yielding a mixture of smaller peptides.

Alternatively, the step of isolating the protein from its vegetable and/or grain source may be eliminated if the isolated protein is commercially available. For enzymatic hydrolysis, the isolated protein is prepared as a solution of 5-25% protein in water, then mixed with a proteolytic enzyme or a combination of enzymes added sequentially, at a ratio of 1:100-1:10 (based on the strength of the enzyme). The solution is then warmed to about 50 to 60° C. The reaction temperature may be adjusted to a lower temperature, which requires increasing the reaction time. Higher reaction temperatures may be used at the risk of approaching the deactivation temperature of the enzyme(s). The pH is adjusted to a pH suitable for the optimal activity and efficacy of the enzyme (in the case of the use of sequential enzymes, sequential pH adjustment is applied). Following hydrolysis, the enzyme(s) are deactivated, for example, by either lowering the pH to an acidic value such as below 5 or increasing the temperature to above about 70° C. Subsequently, the solution is centrifuged or filtered to remove the insoluble material or pellets. Finally, the hydrolyzed proteins are obtained in the dry form through removal of water under reduced pressure and high temperature, or by freeze-drying. Although the resulting protein hydrolysate mixture is an effective antioxidant by itself, at low, effective doses, without impairing the taste and color of a food application; optionally, fractions of the hydrolyzed protein can be further separated by ultrafiltration and/or desalination. The hydrolyzed proteins may be added directly to the water phase of a food system. Alternatively, a carrier such as glycerin, alkylene glycol can be added during the removal of water, reducing the water presence to a maximum of about 5% or below (unfavorable for microorganisms), which then can be added directly to a food system.

The hydrolyzed proteins of the invention surprisingly demonstrate high metal chelating activity. The hydrolyzed proteins of the invention, prepared according to the methods described herein, did not exhibit any notable radical scavenging potential by the DPPH method, the most common test for radical scavenging activity.

The hydrolyzed proteins of the invention require no necessary purification steps, such as ultrafiltration or desalination, nor fractionation of the peptides into peptide fractions with distinct molecular weights and high purity. The crude hydrolysate solution (post enzymatic hydrolysis) unexpectedly, and advantageously, exhibits high activity without any further costly processing. However, purified fractions with lower molecular weights can potentially exhibit higher chelating activity, and possibly superior characteristics in certain applications.

It is surprising and completely unpredicted that a pea protein hydrolysate composition demonstrates high metal chelating activity and provides a stabilizing effect at very low doses in foods. Our surprising results showed that the antioxidant property in food of the crude extract, allows its usage at a very low dose, hence, bypassing any undesired color or flavor effect that might arise from the use of the hydrolyzed proteins as food preservatives.

The metal chelating effects of the compositions of the invention have been identified in all the compositions described, as shown by the results of the ferrozine assay as shown in Table 1. The mechanism by which the compositions of the invention exert antioxidative effects have been demonstrated using model screening systems such as the Ferrozine Assay, which measures the ability of a compound to bind to ferrous iron (Fe²⁺), and the DPPH (2,2-diphenyl-1-picrylhydrazyl) test, which measures the radical scavenging ability of compositions by measuring the ability to bleach the diphenylpicryl hydrazyl radical.

The antioxidant effects of the extracts of the invention, and, optionally, their combinations with other natural and/or synthetic antioxidants, have also been evaluated in simple food models and in actual food/beverage applications. The pH of the food application allows the presence of the peptides in the ionized form to exert potent chelating of pro-oxidant transition metal ions. The chelating of pro-oxidant transition metal ions is useful in food, beverage, nutritional supplement and/or cosmetic applications to stabilize the fresh flavor and prevent the formation of off-flavors.

Another feature of the present invention involves the combination of chelating compositions derived from herbs, spices, fruits and/or vegetables with other natural antioxidants, including, but not limited to, tocopherols, tocotrienols, ascorbic acid, ascorbates, natural gallates, catechins, epigallocatechin gallate, grape seed extract, olive leaf extract, resveratrol, carbazoles, erythorbic acid, erythorbates, carnosol, carnosic acid, rosmarinic acid, rosmanol, xanthohumol, rosemary extract, sage extract, oregano extract, and other spice and herb extracts wherein the majority of the antioxidant activity is due to the presence of radical scavenging agents. By carefully blending materials, it is possible to create antioxidant formulations that contain a complete contingent of oil soluble or dispersible radical scavenging agents, water soluble or dispersible radical scavenging agents, oil soluble or dispersible chelating agents, and water soluble or dispersible chelating agents, or any combination thereof. In this way the antioxidative elements of the composition can be more effectively delivered to the various polar, non-polar and intermediate polarity phases found in multiphase foods, cosmetics, beverages or nutritional supplements.

Less preferably, another feature of the present invention involves the combination of metal chelating compositions derived from herbs, spices, fruits and/or vegetables with synthetic antioxidants such as propyl gallate, BHA, BHT, ethoxyquin, TROLOX®, TBHQ, ascorbyl palmitate, and EDTA. While these compositions are not as preferred as their all-natural counterparts, they are contemplated in combination with the compositions of the present invention.

Another feature of the present invention involves the use of the metal chelating compositions, alone, or in combination with other natural or synthetic antioxidants in the stabilization of foods, beverages, cosmetics and nutritional supplements.

Another feature of the present invention involves foods, beverages, cosmetics, and nutritional supplements treated with the metal chelating compositions, alone, or in combination with other natural or synthetic antioxidants.

The instant protein hydrolysate compositions may be added directly to foods according to the solubility characteristics. They may be dissolved in a carrier, such as an alkylene glycol, glycerin, food grade surfactants, benzyl alcohol, and the like, and then added to foods. They can be dispersed onto solid carriers, such as salt, flour, sugars, maltodextrin, silica (such as CABOSIL®), cyclodextrins, starches, gelatins, lactose, whey powders, proteins, and the like and then added to foods.

The instant protein hydrolysate compositions may be added to cosmetics. By cosmetics we include as examples, but are not limited to:

Lip balm, Lip gloss, lipstick, lip stains, lip tint, blush, bronzers & highlighters, concealers & neutralizers, foundations, foundation primer, glimmers & shimmers, powders, eye shadow, eye color, eye liner, mascara, nail polish, nail treatments-strengtheners, make-up, body creams, moisturizers, suntan preparations, sunless tan formulations, body butter, body scrubs, make-up remover, shampoos, conditioners, dandruff control formulations, anti frizz formulations, straightening formulations, volumizing formulations, styling aids, hairsprays, hair gels, hair colors and tinting formulations, anti-aging creams, body gels, essential oils, creams, cleansers, soaps.

The instant protein hydrolysate compositions may be added to beverages. By beverages we include as examples, but are not limited to: beer, wine, teas, herbal tea, coffee, cappuccino, espresso, café au lait, frappes, lattes, soft drinks (carbonated and still), fruit juices, vegetable juices, milks, lemonades, punches, chocolates, ciders, chai, dairy beverages, smoothies, energy drinks, alcoholic beverages, brandies, gin, vodka, fortified waters, flavored waters, whiskey, distilled spirits, bourbon, malt liquor.

The instant protein hydrolysate compositions may be added to foods, including animal foods. By foods we mean both human and animal foods. By human foods we include as examples, but are not limited to: meat (wild and domestic; fresh and cured, processed and unprocessed, dried, canned), Poultry, fish, vegetable protein, dairy products (milk, cheese, yogurt, ice cream), ground spices, vegetables, pickles, mayonnaise, sauces (pasta sauces, tomato based sauces), salad dressings, dried fruits, nuts, potato flakes, soups, baked goods (breads, pastries, pie crusts, rolls, cookies, crackers, cakes, pies, bagels), vegetable oils, frying oil, fried foods (potato chips, corn chips), prepared cereals (breakfast cereals), cereal grain meals, condiments (ketchup, mustard, cocktail sauce, candies, confectionary, chocolates, baby foods).

By animal foods we include as examples, but are not limited to: extruded pet food, kibbles, dry pet food, semi-dry pet food, and wet pet food.

The instant protein hydrolysate compositions may be added to nutritional supplements. By nutritional supplements we include as examples, but are not limited to: eye health supplements, vitamins, nutrition boosters, carotenoid supplements, protein supplements, energy bars, nutritional bars, algal oils, fish oils, and oils containing polyunsaturated fatty acids.

By metal ions, we mean those metal ions that promote or initiate lipid or other oxidation processes, including, but not limited to Fe²⁺, Fe³⁺, Cu¹⁺, Cu²⁺, and Ni²⁺.

In summary, the present invention comprises:

A natural antioxidant composition comprising hydrolyzed protein derived from a vegetable source, such a

natural antioxidant composition which exhibits metal chelating activity, such a

natural antioxidant composition which is a food preservative, such a

natural antioxidant composition wherein the hydrolyzed protein is a hypo-allergenic protein, such a

natural antioxidant composition wherein the vegetable source is selected from pea and potato, such a

natural antioxidant composition wherein the hydrolyzed protein is a pea protein concentrate, such a

natural antioxidant composition wherein the hydrolyzed protein is obtained by enzymatically hydrolyzing a protein derived from a vegetable source using at least one naturally-derived endopeptidase enzyme, heat inactivating the enzyme, centrifuging or microfiltering the hydrolysate, optionally ultrafiltering the hydrolysate, collecting the hydrolysate, evaporating the hydrolysate to dryness, and optionally, replacing the water with a food carrier, such a

natural antioxidant composition further comprising one or more non-chelating antioxidant components derived from edible spices, fruits and/or vegetables, such a

natural antioxidant composition wherein the non-chelating antioxidant components are selected from tocopherols, tocotrienols, rosemary extract, carnosic acid, carnosol, rosmarinic acid, green tea extract, oregano extract, ascorbic acid, and/or mixtures thereof, such a

natural antioxidant composition further comprising one or more synthetic food grade antioxidants, such a

natural antioxidant composition further comprising chelators, radical scavengers, oxygen scavengers, secondary antioxidants, quenchers and/or antioxidants regenerators derived from natural and/or synthetic sources, such a

method for stabilizing foods, beverages, cosmetics and/or nutritional supplements comprising incorporating the natural antioxidant composition into the food, beverage, cosmetic and/or nutritional supplement in an amount effective to stabilize the fresh flavor and prevent the formation of off-flavors, such a

method comprising incorporating additional natural and/or synthetic antioxidants into the food, beverage, cosmetic and/or nutritional supplement.

### EXAMPLES

### Example 1

### Enzymatic Hydrolysis of Isolated Vegetable Proteins (with ALCALASE® 2.4 L).

Yellow pea protein isolate was weighed (200 g) into a vessel and ten times the weight of water was added to the vessel. The contents were then stirred and heated to 50° C. The pH was monitored and adjusted to within a range of 8.0 to 8.6 with a solution of 45% potassium hydroxide (KOH). After the temperature and pH were stable, ALCALASE® 2.4 L (Novozymes NS) was added at a 1:100 enzyme:substrate (v/w) ratio. The pH was monitored and adjusted with KOH to keep it within a range of 8.0 to 8.6. The hydrolysis was allowed to proceed until the pH reached a stable value and the mixture no longer needed the addition of KOH. The hydrolysis was stopped by heating the mixture to 80° C. for 5 minutes to denature the enzyme. The mixture was then removed from the heat and allowed to cool to room temperature. The mixture was then centrifuged at approximately 3000×g for 3 hours. The supernatant was decanted and the water was removed under heat and vacuum until approximately 30-60% water remained. The amount of solids was determined by subtracting the amount of water in the supernatant. Then, an amount of glycerin or propylene glycol equal to that of the hydrolysate was added. The remaining water was further removed under heat and vacuum to below 1%.

### Example 2

### Showing an Example of a Hydrolysis with a Different Enzyme (Trypsin)

Yellow pea protein isolate was weighed (200 g) into a vessel with sufficient volume to hold all materials. Next, ten times the weight of water was added to the vessel. The contents were then stirred and heated to 50° C. The pH was monitored and adjusted to within a range of 8.0 to 8.6 with a solution of 45% potassium hydroxide (KOH). After the temperature and pH was stable, trypsin was added at a 1:100 enzyme:substrate (v/w) ratio. The pH was monitored and adjusted with KOH to keep it within a range of 8.0 to 8.6. The hydrolysis was allowed to proceed until the pH reached a stable value and the mixture no longer needed the addition of KOH. The hydrolysis was stopped by heating the mixture to 80° C. for 5 minutes to denature the enzyme. The mixture was then removed from the heat and allowed to cool to room temperature. The mixture was then centrifuged at approximately 3000×g for 3 hours. The supernatant was decanted and the water was removed under heat and vacuum until dry.

### Example 3

### Metal Chelation Screening Assay—Ferrozine Assay

A 10,000 ppm stock solution of the yellow pea hydrolysate was made by weighing 100 mg of the hydrolysate and dissolving it in 10 mL of dH₂0 or MeOH. Solutions of ferrozine and iron sulfate heptahydrate (FeS0₄) were made in dH₂O at concentrations of 2 mM and 5 mM respectively. Working solutions of the hydrolysates were made in duplicate by diluting the stock in MeOH or H₂0. A control of MeOH or H₂0 without hydrolysate was included. Blanks, to measure background absorbance, were diluted to the same concentration in the same manner. First, 167 μL of the FeS0₄ solution was added to the control and shaken vigorously by hand 20 times. Second, 335 μL of the ferrozine solution was added the samples and shaken vigorously by hand ten times. Each sample was then subsequently treated in the same manner. The blanks had neither of the solutions added. The samples were then allowed to incubate at room temperature for ten minutes. Next, a spectrophotometer set to read at 562 nm was blanked with MeOH or H₂0. The absorbance of each sample, the control, and each blank was obtained. The percent of the iron chelated by the hydrolysate as compared to the ferrozine control was calculated. Results were initially expressed as % ferrozine inhibited, then converted to an equivalent amount of EDTA that generates the same extent of ferrozine inhibition (results=eq gram EDTA/gram hydrolysate). The results are shown in Table 1.

### Example 4

### DPPH Radical Scavenging Screening Assay

DPPH (2,2-diphenyl-1-picrylhydrazyl) stock solution was prepared by dissolving 38-40 mg of DPPH in 100 mL of MeOH to yield a 1 mM solution. The DPPH solution was sonicated to insure complete dissolution and was prepared fresh the day it was used. Stock solutions of the protein hydrolysates at a 10,000 ppm concentration were prepared by dissolving 0.1 g of each dry hydrolysate in 1.0 mL of deionized water. The resulting mixtures were sonicated to insure complete dissolution. Working solutions of 100 ppm and 1,000 ppm concentration of protein hydrolysate were prepared by adding 100 μL or 1 ml of the 10,000 ppm stock solutions to 9.9 ml or 9.0 mL of MeOH, respectively. 10 mL of each of the 100 ppm or 1000 ppm working solutions was combined with 1.0 mL of the DPPH solution and incubated at room temperature for 10 minutes. The spectral background of the spectrophotometer was zeroed using HPLC grade MeOH, and the absorbance of the extract solutions with added DPPH was measured at 515 nm. The absorbance of the control (1.0 mL of the DPPH solution added to 10 mL MeOH) was measured at 515 nm as well. A percent DPPH inhibition was determined for each of the extracts at the concentration at which it was tested as follows: % DPPH inhibition=(1—(A_{hydrolysate}/A₀)×100, A_{hydrolysate} being the absorbance at 515 nm of the hydrolysate after reaction with DPPH and incubation for 10 minutes at room temperature and A₀ being the absorbance at 515 nm of the control. Results were initially expressed as % DPPH inhibited, then converted to equivalent TROLOX (results=eq gram/g hydrolysate) The DPPH assay results are shown in Table 1.

TABLE 1
Yield, DPPH, Ferrozine and Polarity Test Results.

| | EDTA | TROLOX ® | Yield of |
|---|---|---|---|
| RM | eq | eq | hydrolysis |
| Yellow Pea | 0.694 | 0 | 81.41% |
| (ALCALASE ® 2.4 L) | | | |
| Rice | 0.242 | 0 | 69.84% |
| (Trypsin) | | | |
| Chickpea | 0.260 | 0 | 17.86%* |
| (ALCALASE ® 2.4 L) | | | |
| Potato | 0.120 | 0 | 30.78% |
| (ALCALASE ® 2.4 L) | | | |

*Overall yield of isolating the protein from then flour then hydrolyzing the protein isolate

### Example 5

### Oil in Water (O/W) Emulsion Screening Test

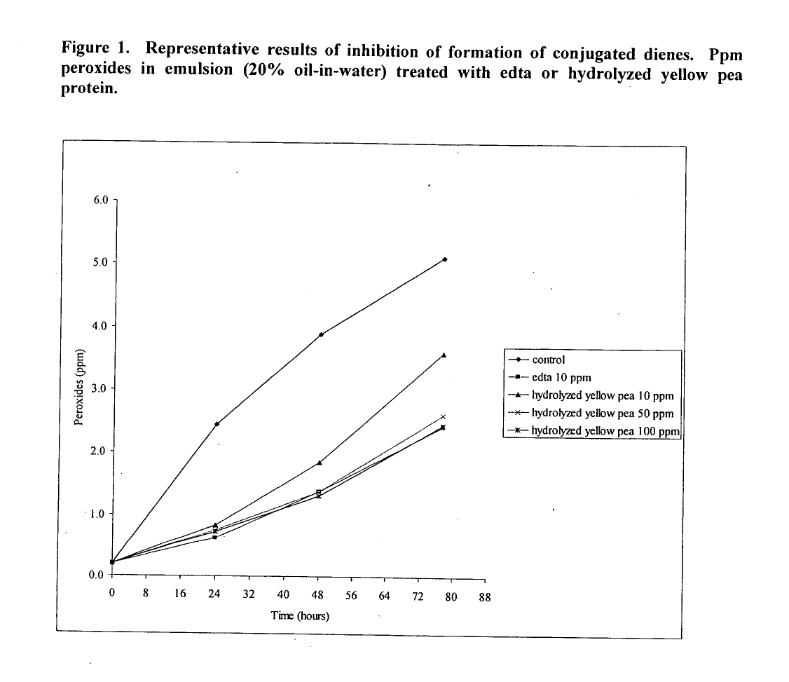

100 g of unfortified canola oil was mixed with 400 mL of deionized water and 10 g of polysorbate-20 (Tween 20) using a WARING® blender. The blended emulsion was passed through a PVA single-stage homogenizer 10 times, and then stored refrigerated at 60° C. 10,000 ppm stock solutions of antioxidant extracts were made by dissolving 0.1 g of the antioxidant extracts in 1.0 mL of deionized water and sonicating for 10 minutes. A 1,000 ppm stock solution of EDTA was made by dissolving 0.01 g of EDTA in 1.0 mL of deionized water. Emulsion solutions containing various concentrations of antioxidant extracts were prepared and incubated at 60° C. on an orbital shaker along with control treatments without antioxidants. Measurements were taken once a day, for seven consecutive days by measuring out 20 μL of each emulsion treatment into 10 mL of 2-propanol. The UV absorbance of the conjugated dienes was measured at 234 nm. Experiments were done in triplicate. For each antioxidant/extract treated emulsion solution, the absorbance (at A=234 nm) was plotted against time. The results are shown in FIG. 1.

### Example 6

### Water in Oil (W/O) Emulsion Screening Test

The following general recipe was used to produce margarine:

| | |
|---|---|
| Ingredient | Amount |

Fat Phase

| | |
|---|---|
| Hydroxylated Soy Lecithin | 1% |
| Glycerol mono-oleate | 1% |
| β-Carotene | 10 ppm |
| Diacetyl | 10 ppm |
| Palm Oil | 55% |
| Soybean Oil | 25% |

Aqueous Phase

| | |
|---|---|
| Water | 17% |
| Salt (NaCl) | 1% |

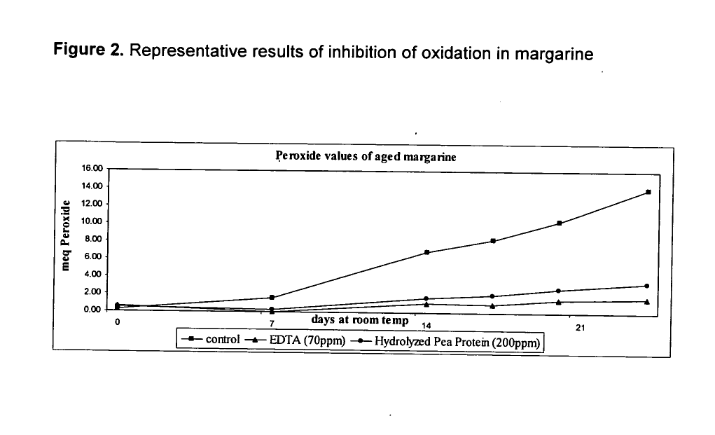

Three different margarine samples were prepared, containing: EDTA (70 ppm), hydrolyzed pea protein (200 ppm), and no antioxidants (control), and incubated at 22-23° C., in the dark. Margarine samples of each treatment were pulled periodically, and the fat was separated by melting at 60° C., followed by centrifuging at ˜1,000 g and decanting the upper (fat) phase. Oxidation was evaluated by measuring the peroxide value according to the AOCS official method Cd 8b-90, which was plotted against time. The results are shown in FIG. 2. Results showed that the hydrolyzed pea protein inhibited oxidation in comparison to the untreated control, reflecting in lower levels of peroxide value (PV) over time.

### Example 7

### Antioxidant Activity in Powdered Milk

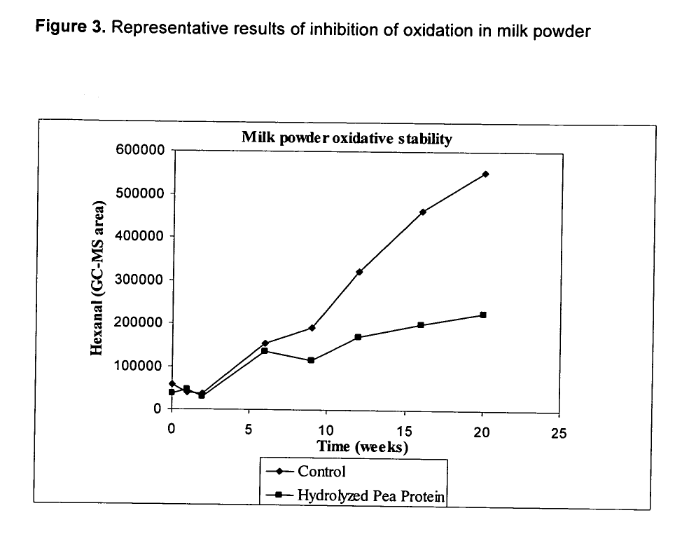

Fresh milk was treated with hydrolyzed pea protein at 100 ppm, homogenized, spray-dryed, then incubated at room temperature (22-23° C.), in the dark, in comparison to the same spray-dried milk without any additives (control). Samples were analyzed periodically by gas chromatography, and oxidation was traced by monitoring the generation and accumulation of secondary oxidation product (hexanal). Representative results are shown in FIG. 3. The experiment showed an antioxidative protective effect of the hydrolyzed pea protein, reflected in lower levels of hexanal.

### Example 8

### Antioxidant Activity in Extruded Corn Cereal

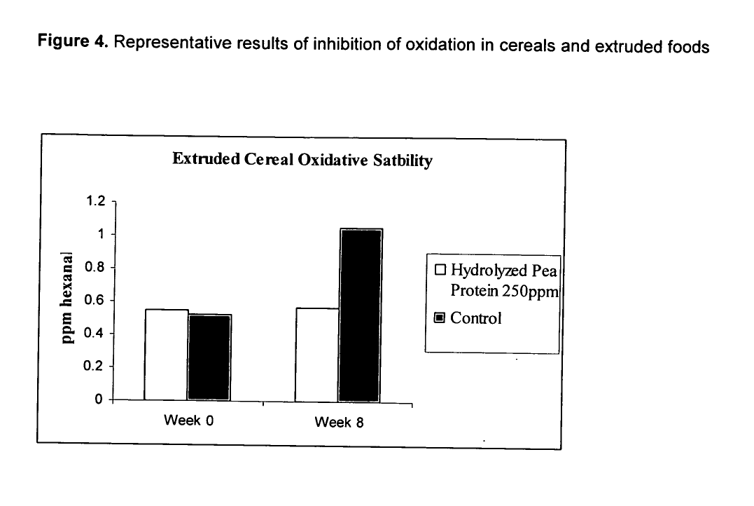

Extruded breakfast corn cereal was prepared using a recipe consisting of 5% milled flaxseed and 95% corn semolina, and 250 ppm of hydrolyzed pea protein, in comparison to the same recipe without any antioxidant additives (control). Samples were packaged and incubated in the dark at room temperature (22-23° C.) for 8 weeks. The extruded cereal sample containing was more oxidatively stable as it exhibited lower levels of the oxidation marker hexanal (detected by GC). Representative results are shown in FIG. 4.

### REFERENCES

Oviedo, C.; Rodriguez, J. (2003) Quim. Nova 26(6), 901-905.
Bou, R.; Codony, R.; Tres, A.; Decker, E. A.; Guardiola, F. (2008) Analytical Biochemistry 377, 1-15.
Carter, P. (1971) Analytical Biochemistry 40, 450-458.
8zyrn˜iewicz, A.; Jedrychowski, L. (2008) Polish Journal of Food and Nutritional Sciences 58(3), 345-350.
Hurniski, L. M.; Aluko, R. E. (2007) J. of Food Science 72(8), 8605-8611.
Aluko, R. E. (2008) J. of AOAC International 91(4), 947-956.
Pownall, T. L.; Udenigwe; C. C.; Aluko, R. E. (2010) J. Agric. and Food Chem. 58)8), 4712-4718.

1. A natural antioxidant composition comprising hydrolyzed protein derived from a vegetable source.

2. The natural antioxidant composition of claim 1 which exhibits metal chelating activity.

3. The natural antioxidant composition of claim 1 which is a food preservative.

4. The natural antioxidant composition of claim 1, wherein the hydrolyzed protein is a hypo-allergenic protein.

5. The natural antioxidant composition of claim 1, wherein the vegetable source is selected from pea and potato.

6. The natural antioxidant composition of claim 1, wherein the hydrolyzed protein is derived from a pea protein concentrate.

7. The natural antioxidant composition of claim 1, wherein the hydrolyzed protein is obtained by enzymatically hydrolyzing a protein derived from a vegetable source using at least one naturally-derived endopeptidase enzyme, heat inactivating the enzyme, centrifuging or microfiltering the hydrolysate, optionally ultrafiltering the hydrolysate, collecting the hydrolysate, evaporating the hydrolysate to dryness, and optionally, replacing the water with a food carrier.

8. The natural antioxidant composition of claim 1, further comprising one or more non-chelating antioxidant components derived from edible spices, fruits and/or vegetables.

9. The natural antioxidant composition of claim 8, wherein the non-chelating antioxidant components are selected from tocopherols, tocotrienols, rosemary extract, carnosic acid, carnosol, rosmarinic acid, green tea extract, oregano extract, ascorbic acid, and/or mixtures thereof.

10. The natural antioxidant composition of claim 1, further comprising one or more synthetic food grade antioxidants.

11. The natural antioxidant composition of claim 1, further comprising chelators, radical scavengers, oxygen scavengers, secondary antioxidants, quenchers and/or antioxidants regenerators derived from natural and/or synthetic sources.

12. A method for stabilizing foods, beverages, cosmetics and/or nutritional supplements comprising incorporating the natural antioxidant composition of claim 1 into the food, beverage, cosmetic and/or nutritional supplement in an amount effective to stabilize the fresh flavor and prevent the formation of off-flavors.

13. The method of claim 12, comprising incorporating additional natural and/or synthetic antioxidants into the food, beverage, cosmetic and/or nutritional supplement.
